(19) United States
(12) Patent Application Publication
(10) Pub. No.: US 2023/0086119 A1
(43) Pub. Date: Mar. 23, 2023

(54) ANTICANCER AGENT CONTAINING RHOA PEPTIDE INHIBITOR AS AN ACTIVE INGREDIENT

(71) Applicant: EL MED, Chuncheon-si (KR)

(72) Inventors: Jae Bong PARK, Chuncheon-si (KR); Jae Gyu KIM, Chuncheon-si (KR)

(21) Appl. No.: 17/791,202

(22) PCT Filed: Dec. 31, 2020
(86) PCT No.: PCT/KR2020/019481

§ 371 Date: Jul. 6, 2022

(30) Foreign Application Priority Data
Nov. 13, 2020 (KR) 10-2020-0151789

(51) Int. Cl. A61K 38/07; A61K 38/10; A61P 35/00

(52) U.S. Cl. CPC A61K 38/07; A61K 38/10; A61P 35/00

(57) ABSTRACT

Provided is an anticancer drug containing a RhoA peptide inhibitor as an active ingredient.

### BACKGROUND OF THE DISCLOSURE

### Field of the Disclosure

The present disclosure relates to an anticancer drug containing a RhoA peptide inhibitor as an active ingredient.

### Related Art

Wnt signaling regulates a variety of biological processes including cell proliferation, differentiation, polarity, survival and development, and migration to oncogenic events. Wnt signaling can be classified into two pathways: a standard modified β-catenin-dependent pathway and a non-standard non-transformed β-catenin-independent pathway (Doble and Woodgett, 2003). In the absence of a Wnt signal, glycogen synthase kinase-β (GSK-3β) phosphorylates β-catenin in the “destructive complex”. This complex consists of GSK-3β, adenomatous polyposis coli (APC), Axin, β-catenin, and casein kinase I (CM), in which CM primes by the Ser45 phosphorylation of β-catenin and GSK-3β subsequently phosphorylates Thr41, Ser37 and Ser33 of β-catenin, leading to its degradation by 26S proteasome (Doble and Woodgett, 2003).

In the presence of Wnt, GSK-3β phosphorylates lipoprotein receptor-associated protein 6 (LRP6) rather than β-catenin, and thus there is a cellular stimulation in the standard pathway required for axin to bind to LRP6 along with GSK-3β and APC (McCubrey et al., 2014). The phosphorylated N-terminus of LRP6 by Wnt stimulation binds to GSK-3β and thereby inhibits the ability of GSK-3β to phosphorylate β-catenin (Stamos et al., 2014). (3-Catenin is released from GSK-3β, increases its stability, and accumulates in the cytoplasm and nucleus (Wu and Pan, 2010).

When binding to a Wnt ligand, the Wnt receptor Frizzled binds to a disheveled (Dv1) protein, which binds to Rho-GTPase through Dv1-associated activator 1 (Daaml), which is a formin homology adapter protein.

Interestingly, Wnt3A induces both the standard β-catenin-dependent signaling and the non-standard Rho-GTPase-mediated signaling (Bikkavilli et al., 2008; Endo et al., 2005; Kishida et al., 2004; Qiang et al., 2003). Due to Wnt3A, which induces both RhoA activation and β-catenin accumulation, the motility of CHO-Kl cells was not inhibited by disruption of the β-catenin pathway, but was partially blocked by inhibitors of ROCK (Endo et al., 2005). Moreover, β-catenin stabilization or nuclear translocation of (3-catenin by Wnt3A was not affected by RhoA (Rossol-Allison et al., 2009). Therefore, it is possible that β-catenin accumulation and RhoA activation occur independently. However, it was recently reported that β-catenin accumulation relies on RhoA activity in response to Wnt3A. In addition, ROCK1 phosphorylates Ser9 of GSK-3β(Kim et al., 2017b). However, it is not clear whether p-Ser9 GSK-3β is directly associated with β-catenin accumulation in the Wnt3A signaling pathway.

RhoA, being a small GTPase, is activated by several guanine nucleotide exchange factors (GEFs), and GTP binds to RhoA to exhibit activated RhoA. In contrast, RhoA can be inactivated by the GTPase activating protein (GAP) that catalyzes GTP hydrolysis, which leads to GDP-bound RhoA (which is inactive). While the inactive RhoA-GDP is confined to the cytosol in a complex with Rho guanine nucleotide dissociation inhibitor (RhoGDI), the active RhoA-GTP is confined to a membrane through linkage through a geranylgeranyl group covalently attached to a Cys residue at the C-terminus of RhoA. It is noteworthy that IKKγ (also called NEMO) promotes RhoA activation through IKKγ (NEMO) thereby causing dissociation of the RhoA-RhoGDI complex (Kim et al., 2014). The active RhoA-GTP binds to an effector protein including Rho-associated coil kinase (ROCK) and transmits the signal to the downstream signaling pathway (Jaffe and Hall, 2005). In particular, Tyr42 phosphorylated RhoA binds to IKKγ and thereby induces IKKβ activation, Iκβ phosphorylation, and NF-κB activation (Kim et al., 2017a). In irregular Wnt signaling, the RhoA pathway acts as a Dv1 protein, and the activated Daaml binds to weak-similarity GEF (WGEF) and Rho GEF so as to promote the formation of RhoA-GTP, thereby inducing ROCK activation and subsequent dynamic cytoskeletal rearrangement.

In cancer, Rho GTPase is overexpressed in many cases of tumor tissue (Jung et al., 2020). In addition, GEF is upregulated and GAP is downregulated. Effector proteins (e.g., ROCK) are upregulated in malignant tissue. Rho-GAP domain-containing DLC (deleted in liver cancer-1) is downregulated in various tumors and is regarded as a tumor suppressor. Therefore, inhibitors against Rho GTPase, including effector proteins (e.g., RhoA, Cdc42, Rac1, GEF, and ROCK), have been developed to treat various cancers (Cardama et al., 2017). Currently, there are no chemical inhibitors that directly inhibit RhoA activity. C3 exoenzyme can inhibit RhoA, RhoB, and RhoC through ADP-ribosylation at asparagine 41 of RhoA (Aktories et al., 1987; Ohashi and Narumiya, 1987). However, since C3-exoenzyme is a protein, it has a barrier to transport to cells. Instead, the ROCK inhibitor Y27632 has been widely used in the RhoA signaling pathway (Uehata et al., 1997), (Rodrigues et al., 2001). C3 exoenzyme completely abolished the invasion of epithelial cells MDCKts.src and colon cell line PCmsrc induced by intestinal trefoil factor pS2 (Rodrigues et al., 2001). Although controversial, cancer cell death can be mediated by RhoA and ROCK (Li et al., 2014). For this reason, both C3 exo-enzyme and Y27632 were not used as anticancer drugs clinically.

In particular, RhoA G17V (Yoo, 2014, 24584070; Sakata-Yanagimoto, 2014, 24413737; Palomero, 2014, 24413734; Manso, 2014, 24786457) and Y42C mutations are frequently found in T cell lymphoma and diffuse gastric cancer (Kakiuchi et al., 2014). The expression of RhoA G17V increased proliferation in Jurkat cells. However, this is inconsistent with previous reports that overexpression of wild-type RhoA promoted tumorigenesis. Many somatic mutations of ROCK1 and ROCK2 were found (Wei, 2016, 26725045), and it was reported that Y27632 treatment inhibits the transformation of NIH3T3 cells by Dbl and Ras (Sahai et al., 1999). Therefore, there is a constant demand for technology development for a chemical inhibitor that directly inhibits RhoA activity.

### SUMMARY

p-Tyr42 RhoA is closely associated with tumorigenesis of breast cancer cells. Since p-Tyr42 RhoA is important for survival probability, the development of the present inventors was focused on inhibition p-Tyr42 RhoA activity by breaking the binding to the effector protein. In addition, in the present disclosure, the relationship between RhoA activation to Wnt3A and β-catenin accumulation was investigated. The present inventors have found that Wnt3A induces the interaction of p-Tyr42 RhoA with β-catenin and that p-Tyr42 RhoA delivers β-ketenin to the nucleus. The present inventors have also found that p-Tyr42 RhoA and β-catenin regulate tumor formation. Therefore, it was assumed herein that destruction of the complex of p-Tyr42 RhoA and β-catenin will inhibit cancer cell proliferation. Consequently, the present inventors have developed an EM3 drug that inhibits cell proliferation and migration of several cancer cells.

According to an embodiment of the present disclosure, an anticancer drug containing the compound of Formula 1 as an active ingredient is provided.

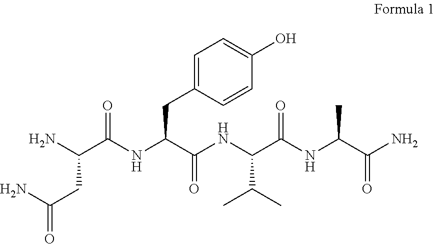 [SMILES: CC(C)[C@H](NC(=O)[C@H](Cc1ccc(O)cc1)NC(=O)[C@@H](N)CC(N)=O)C(=O)N[C@@H](C)C(N)=O]

According to an embodiment of the present disclosure, an anticancer drug containing the compound of Formula 2 below as an active ingredient is provided.

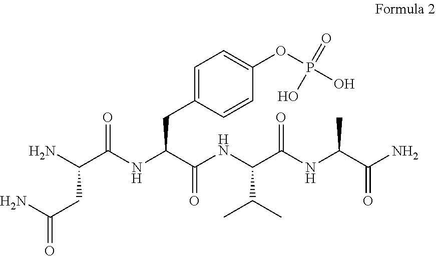 [SMILES: CC(C)[C@H](NC(=O)[C@H](Cc1ccc(OP(=O)(O)O)cc1)NC(=O)[C@@H](N)CC(N)=O)C(=O)N[C@@H](C)C(N)=O]

According to an embodiment of the present disclosure, an anticancer drug containing the compound of Formula 3 as an active ingredient is provided.

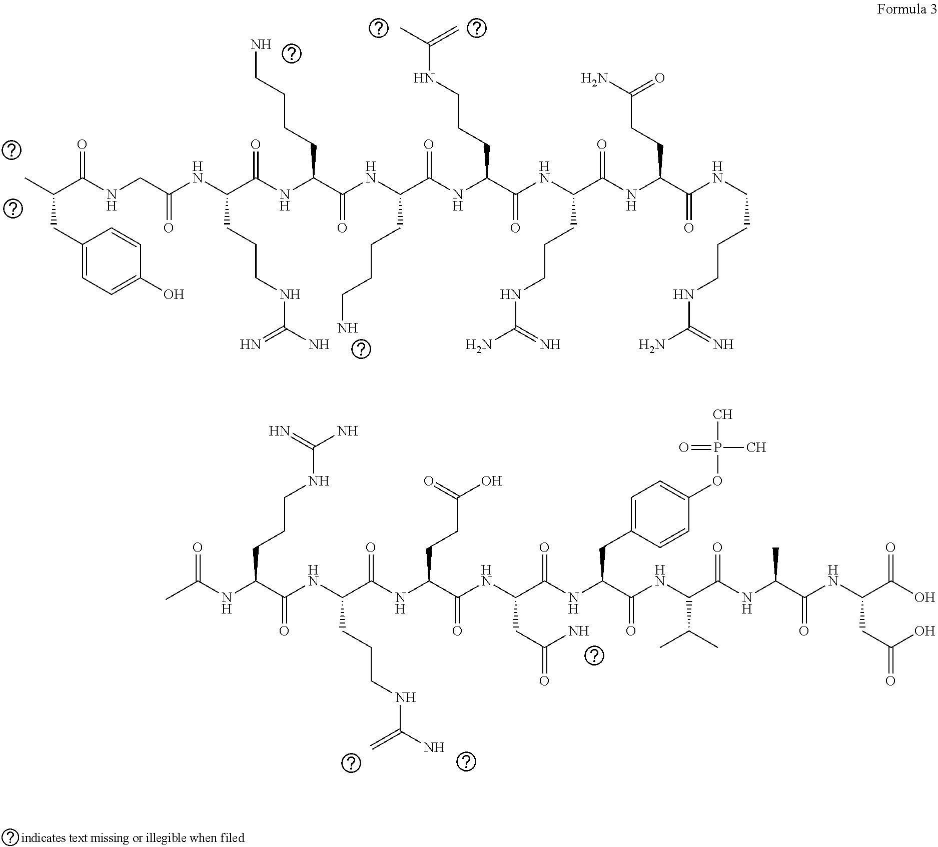

According to an embodiment of the present disclosure, an anticancer drug containing the compound of Formula 4 as an active ingredient is provided.

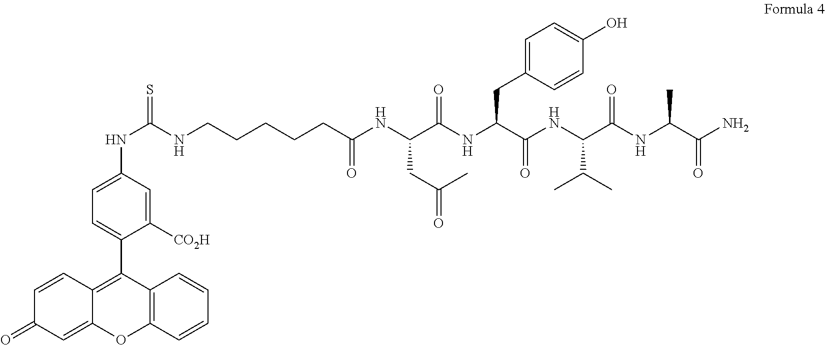 [SMILES: CC(=O)C[C@H](NC(=O)CCCCCNC(=S)Nc1ccc(-c2c3ccc(=O)cc-3oc3ccccc23)c(C(=O)O)c1)C(=O)N[C@@H](Cc1ccc(O)cc1)C(=O)N[C@H](C(=O)N[C@@H](C)C(N)=O)C(C)C]

According to an embodiment of the present disclosure, an anticancer drug containing the compound of Formula 5 as an active ingredient is provided.

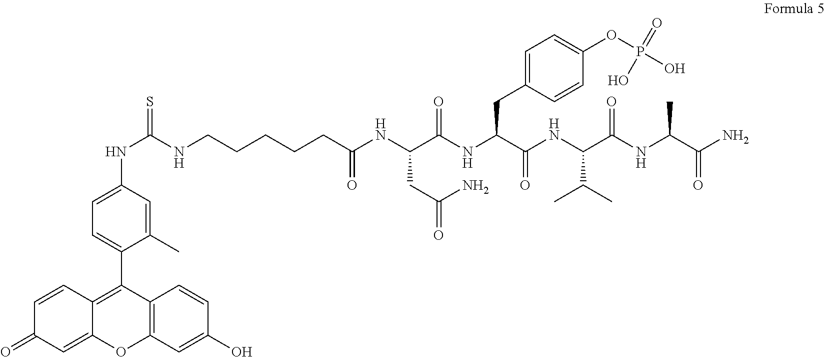 [SMILES: Cc1cc(NC(=S)NCCCCCC(=O)N[C@@H](CC(N)=O)C(=O)N[C@@H](Cc2ccc(OP(=O)(O)O)cc2)C(=O)N[C@H](C(=O)N[C@@H](C)C(N)=O)C(C)C)ccc1-c1c2ccc(=O)cc-2oc2cc(O)ccc12]

According to an embodiment of the present disclosure, an anticancer drug containing the compound of Formula 6 as an active ingredient is provided.

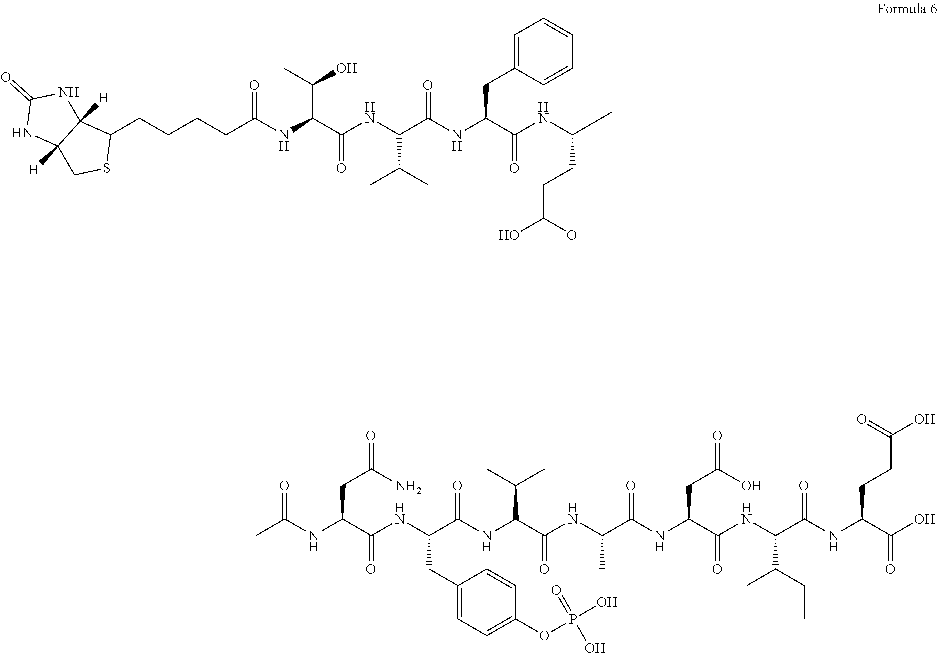

According to an embodiment of the present disclosure, an anticancer drug containing the compound of Formula 7 as an active ingredient is provided.

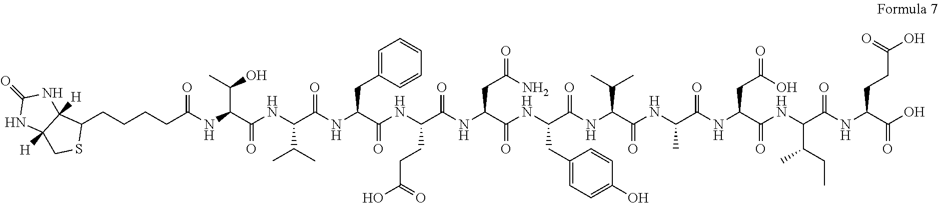 [SMILES: CC[C@H](C)C(NC(=O)[C@H](CC(=O)O)NC(=O)[C@H](C)NC(=O)[C@@H](NC(=O)[C@H](Cc1ccc(O)cc1)NC(=O)[C@H](CC(N)=O)NC(=O)[C@H](CCC(=O)O)NC(=O)[C@H](Cc1ccccc1)NC(=O)[C@@H](NC(=O)[C@@H](NC(=O)CCCCC1SC[C@@H]2NC(=O)N[C@H]12)[C@@H](C)O)C(C)C)C(C)C)C(=O)N[C@@H](CCC(=O)O)C(=O)O]

### Advantageous Effects

The RhoA peptide inhibitor according to the present disclosure has an effect of preventing cancer cell migration.

In addition, the effects of the present disclosure are not limited to the effects mentioned above, and other effects that are not mentioned may be clearly understood by a person skilled in the art from the description set forth hereunder.

### BRIEF DESCRIPTION OF THE DRAWINGS

FIGS. 1 to 4 show clinical results of p-Tyr42 Rho expression in cancer patients.

FIGS. 5 to 10 show that P-Tyr42 RhoA induces cell proliferation in response to Wnt3A.

FIGS. 11 to 21 show the interaction of β-catenin with RhoA in response to Wnt3A.

FIGS. 22-36 show designed RhoA inhibitors.

FIG. 37 shows the inhibition of proliferation of various cancer cells by RhoA peptide inhibitors (PR2, PR3, and PR4).

FIG. 38 shows the inhibition of the proliferation of various cancer cells by the RhoA peptide inhibitors (EM3/E3).

FIGS. 39 to 40 show the effect of the substituted amino acid of EM3 on cell proliferation.

FIGS. 41 to 42 show the prevention of cancer cell migration by RhoA peptide inhibitors.

### DESCRIPTION OF EXEMPLARY EMBODIMENTS

The present disclosure provides an anticancer drug containing the compound of Formula 1 below as an active ingredient.

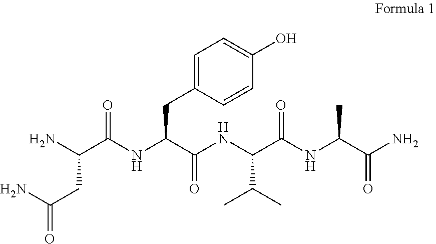 [SMILES: CC(C)[C@H](NC(=O)[C@H](Cc1ccc(O)cc1)NC(=O)[C@@H](N)CC(N)=O)C(=O)N[C@@H](C)C(N)=O]

Hereinafter, the present disclosure will be described in detail. Before going into details, it should be noted that terms or words used in the present specification and claims should not be construed as being limited to their usual or dictionary meanings, but should be interpreted as a meaning and concept complying with the technical idea of the present disclosure on the basis of the principle that it can be appropriately defined so as to describe their own invention in the best way. Therefore, the constitutions described in the embodiments of the present specification are merely the most preferred embodiments of the present disclosure, and do not represent all the technical spirit of the present disclosure. Therefore, it should be understood that there may be various equivalents and modified embodiments thereof that can replace the same at the time of filing the present application.

### Experimental Example

Cell Culture

All cells tested were cultured in Dulbecco's Modified Eagle Medium (DMEM; Weljin, Gyeongsan) containing 5% fetal bovine serum (FBS; GIBCO, Carlsbad, USA) and 1% penicillin-streptomycin (complete medium) under 5% CO2 at 37° C.

Western Blotting

HEK293T cells were collected and washed with ice-cold PBS, and dissolved with RIPA buffer (50 mM Tris-HCl (pH7.4), 150 mM NaCl, 1% Nonidet P-40, 0.25% NaN₃, 1 mM EDTA, 1 mM PMSF, 1 μg/mL aprotinin, 1 μg/mL leupeptin, 1 μg/mL pepstatin, 1 mM sodium orthovanadate, and 1 mM NaF). The cell lysate was centrifuged at 13,475×g for 20 minutes and the supernatant was analyzed for protein concentration using the BCA Protein Assay Kit (Pierce; Rockford, USA). Proteins of the cell lysate were run on SDS-PAGE and transferred to a PVDF membrane (Millipore; Billerica, USA). Anti-RhoA ( 1/500), anti-actin (Santa Cruz; Dallas, Calif.), anti-GSK-3β( 1/500), anti-phospho (Ser9)-GSK-3β( 1/500) (both Cell Signaling, Beverly, USA), anti-O-catenin ( 1/1000) (Invitrogen, Carlsbad, USA), anti-active β-catenin ( 1/500) (Millipore, Billerica, USA), and anti-Tau ( 1/500) (Biosource; Camarillo, Calif.) antibodies were incubated with PVDF membranes at room temperature for 2 hours, and then each was washed 3 times for 5 minutes. Thereafter, the membrane was incubated for 1 hour with anti-rat IgG antibody or anti-mouse IgG antibody conjugated with horse radish peroxidase (HRP) (both provided by ENZO Life Science; Farmingdale, USA, 1/1000 dilution) and each was washed later three times for 10 minutes. The membrane was incubated with an enhanced chemiluminescence (ECL) reagent (Amersham; Uppsala, Sweden) and then exposed to an X-ray film. The strength of the band was quantified with the ImageJ program of the U.S. National Institutes of Health (Bethesda, USA).

Immunoprecipitation (IP)

1×10⁷ Cells were washed with 1×PBS, and the cell lysate was prepared using cell lysis buffer (20 mM Tris pH 7.4, 120 mM NaCl, and 1% Nonidet P-40) containing 10 μg/mL each of leupeptin and aprotinin and each of NaF, Na₃VO₄, PMSF and 5 mM MgCl₂. The lysate was used for immunoprecipitation by specific antibodies.

Purification of Recombinant GST-B-Catenin and GST-Rhoa Protein and Tat-C3 Toxin

Recombinant GST-β-catenin and GST-RhoA were expressed in E. coli using the pGEX-4T1 host vector. Protein expression was induced by adding 0.5 mM isopropylthio-galactosidase (IPTG) to the transformed culture of E. coli. GST-β-catenin and GST-RhoA fusion proteins were purified using glutathione (GSH)-Sepharose 4B beads. To prepare without the GST protein, the GST protein was cleaved and removed according to the manufacturer's protocol using thrombin. Recombinant Tat-C3 toxin was purified from E. coli.

Transfection with RhoA Plasmid

Cells were transfected with RhoA plasmid using the HiPerFect (QIAGEN) transfection reagent. The plasmid (2 μg/mL) in 100 μL culture medium without serum was mixed with 3 μL of the HiPerFect transfection reagent by vortexing for 10 seconds and incubated for 5-10 minutes at room temperature. Then, the mixture was added dropwise to the cells in 5 mL serum-free medium. After 4-5 hours, 8×10⁵ cells were cultured in 60 mm dishes with 5 mL of appropriate culture medium containing serum and antibiotics under 5% CO2 at 37° C. and.

Analysis of Survival Probability of Gastric Cancer Patients

The present inventors determined the relative p-Tyr42 Rho levels in tissues of gastric cancer patients and classified them into two groups: low and high p-Tyr42 Rho expressers. Samples were collected at the Cancer Center of Pusan National University Hospital (Pusan) supported by the National Cancer Center. Information on the death and survival of the patients was obtained from the Korean Statistical Information Service (KOSIS) and approved by the Institutional Review Board (IRB) of Hallym University (Chuncheon).

Statistical Analysis

All experiments were performed at least 3 times, and samples in the assay were analyzed 2 or 3 times. Data were expressed as mean±SE. Statistical comparisons were performed with Student's t-test using the GraphPad Prism program (GraphPad Software, San Diego, USA), and the difference between the two groups was considered statistically significant when the P value was less than the specified limit (*P<0.05, **P<0.01, ***P<0.001).

### Evaluation Examples

Upregulation of Levels of p-Tyr42 RhoA in Gastric Cancer Patients

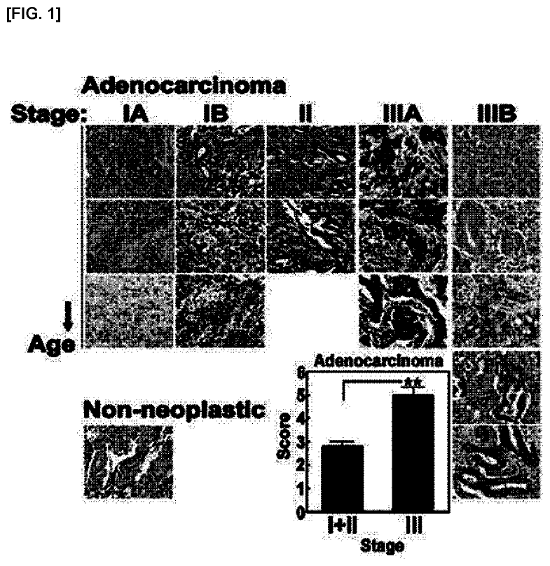

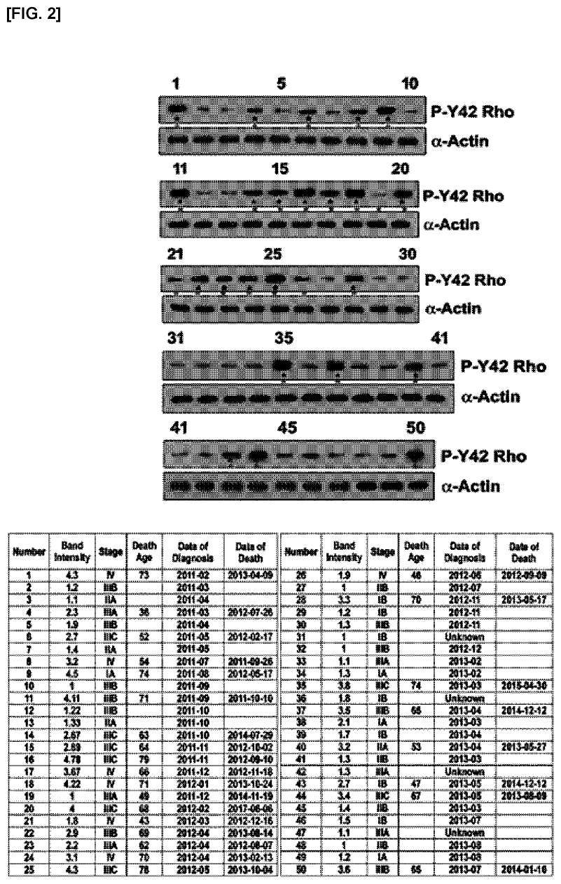

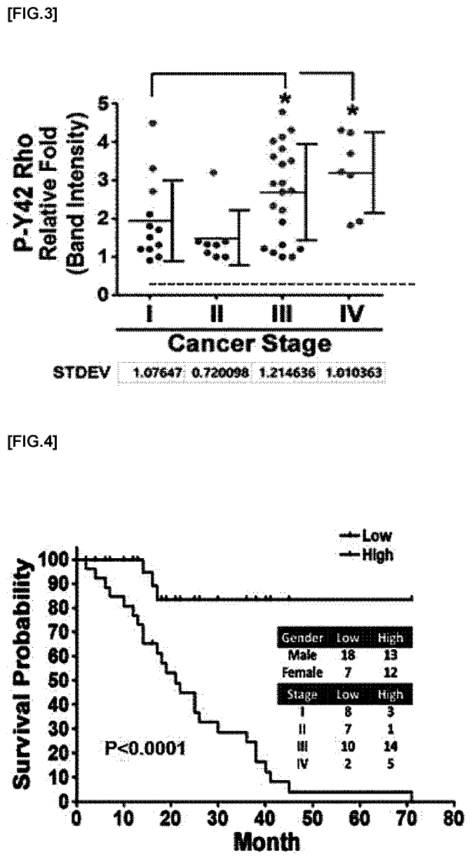

p-Tyr42 Rho levels were significantly increased in more advanced cancer types with data focused on gastric adenocarcinoma cases. In this case, the sample of a gastric adenocarcinoma patient at stage III showed much stronger staining of p-Tyr42 Rho than those at stage I and stage II (FIG. 1). For absolute comparison of samples of various gastric cancer patients, the relative level of p-Tyr42 Rho was determined through western blotting and then correlated with patient survival data (FIG. 2). The p-Tyr42 Rho intensity generally increased, but not strictly, in the more advanced stages of the tumor, almost all patients with surprisingly high p-Tyr42 RhoA levels died significantly (FIG. 3). In addition, patients with high p-Tyr42 Rho GTPase levels showed a much worse survival probability (survival <5%, FIG. 4) at 5 years (60 months) compared to the patients with low p-Tyr42 Rho levels (survival rate>85%). These results indicate that higher levels of p-Tyr42 Rho are very detrimental to survival in gastric tumor samples.

Effect of RhoA and ROCK on Cell Proliferation on Wnt3A

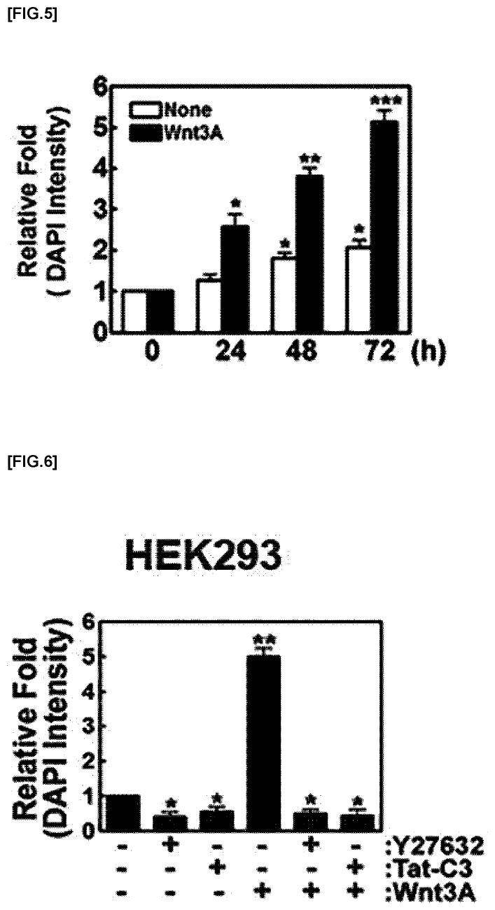

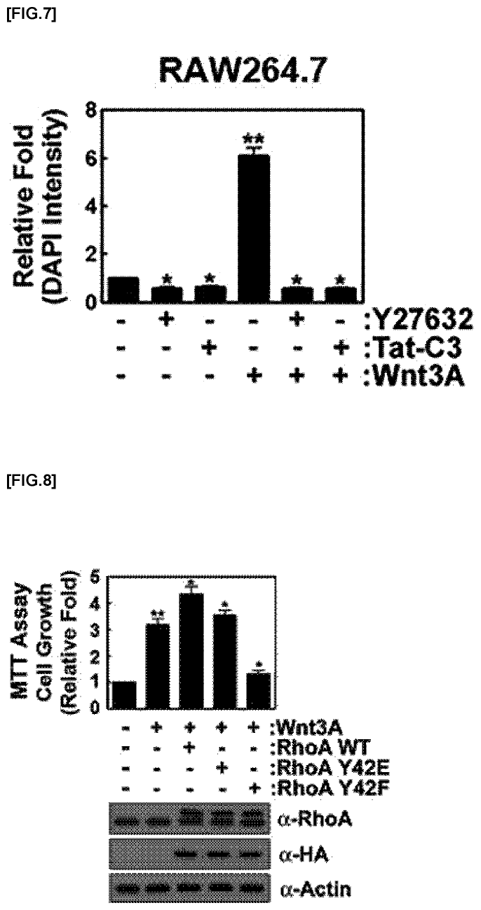

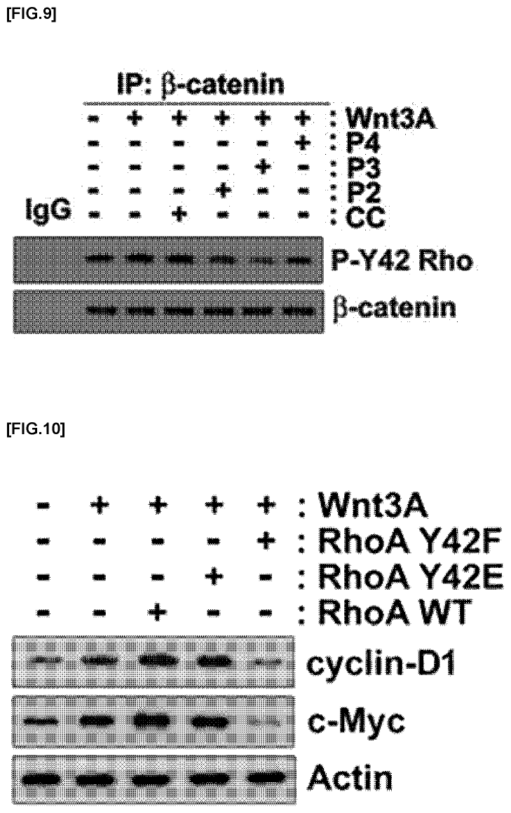

Wnt3A improved the proliferation of HEK293T cells (FIG. 5). However, RhoA inhibitor (Tat-C3) and ROCK inhibitor (Y27632) abolished this proliferation (FIG. 6). Similarly, Tat-C3 and Y27632 also inhibited Wnt3A-mediated proliferation in the macrophage cell line RAW264.7 (FIG. 7). RhoAY42F transfection also significantly removed cell proliferation (FIG. 8). Wnt3A also induced the expression of cyclin D1 and c-Myc in test cells, but Tat-C3 and Y27632 abolished this increased expression of Wnt3A (FIG. 9). While RhoA WT- and RhoAY42E-transfected cells also increased the expression of cyclin D1 and c-Myc in the presence of Wnt3A, RhoAY42F transfection inhibited this upregulation of cyclin D1 and c-Myc (FIG. 10).

Formation of RhoA/r3-Catenin Complex with Wnt3A Stimulation

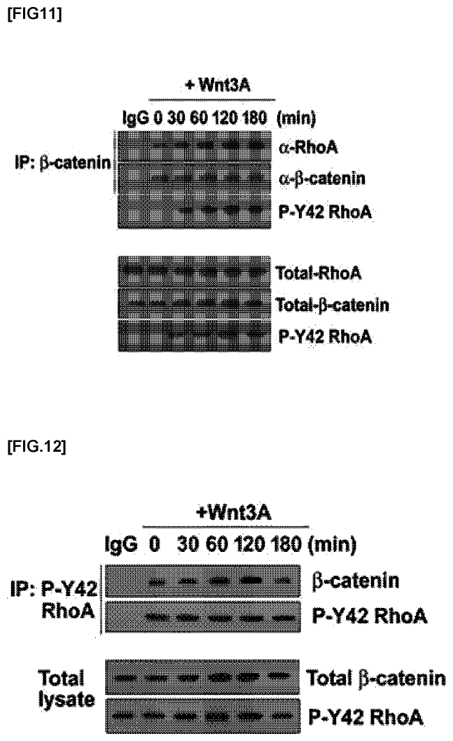

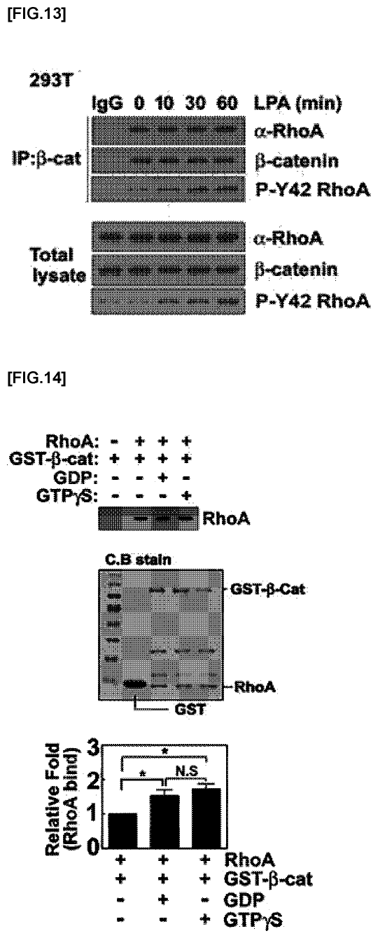

The co-immunoprecipitation of β-catenin with RhoA in Wnt3A was observed (FIG. 11). In turn, p-Tyr42 Rho was co-immunoprecipitated with β-catenin (FIG. 12). Since it has been reported that lysophosphatidic acid (LPA) to activate β-catenin along with nuclear translocation of β-catenin (Burkhalter et al., 2015; Sun et al., 2013), the possibility of complex formation of β-catenin/RhoA or p-Tyr42 Rho by LPA was examined. LPA did not change the levels of RhoA and β-catenin, but p-Tyr42 Rho levels apparently increased (FIG. 13). Likewise, co-immunoprecipitation of RhoA and β-catenin was apparently shown, but the relative level of the co-immunoprecipitation was almost not changed. However, the association between β-catenin and p-Tyr42 Rho revealed through co-immunoprecipitation was significantly increased by LPA treatment (FIG. 13). Next, the present inventors have attempted to determine whether the GDP/GTP binding status of RhoA determines the interaction between RhoA and β-catenin. Recombinant GST-β-catenin that binds to RhoA-GTPγS or RhoA-GDP in vitro was not significantly different (FIG. 14), and this implies that the binding of GDP or GTP to RhoA is not important for the interaction between RhoA and β-catenin.

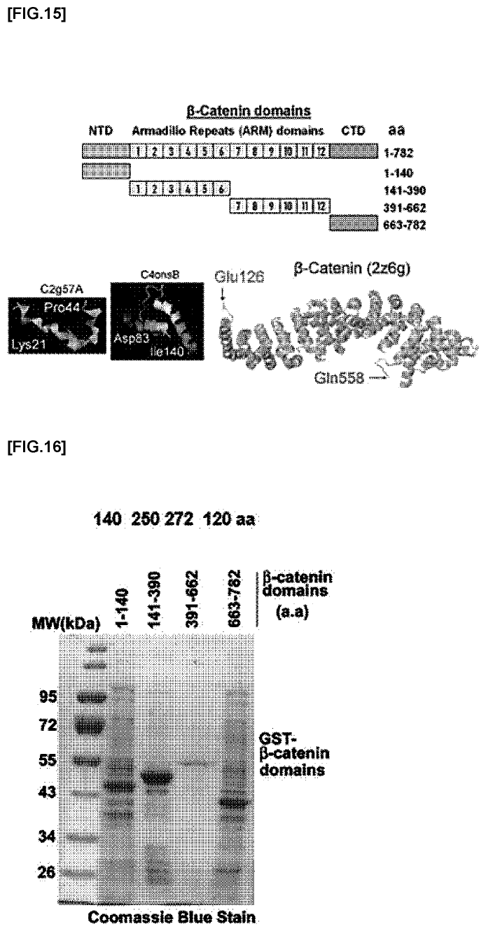

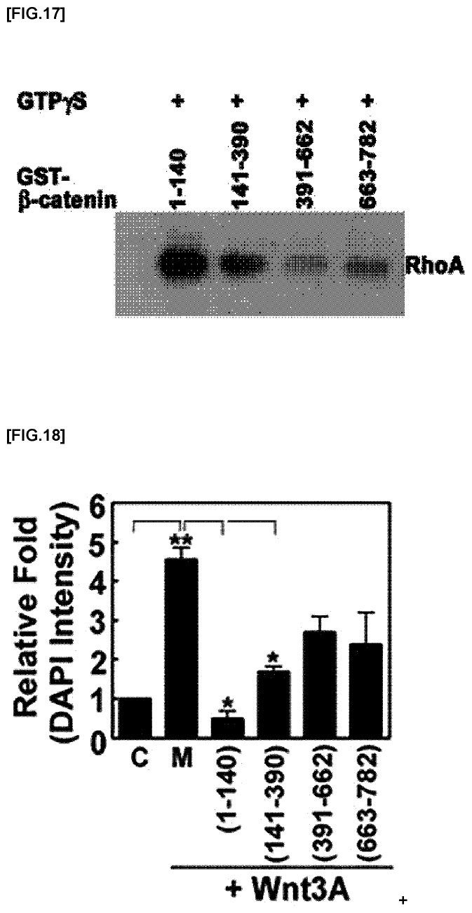

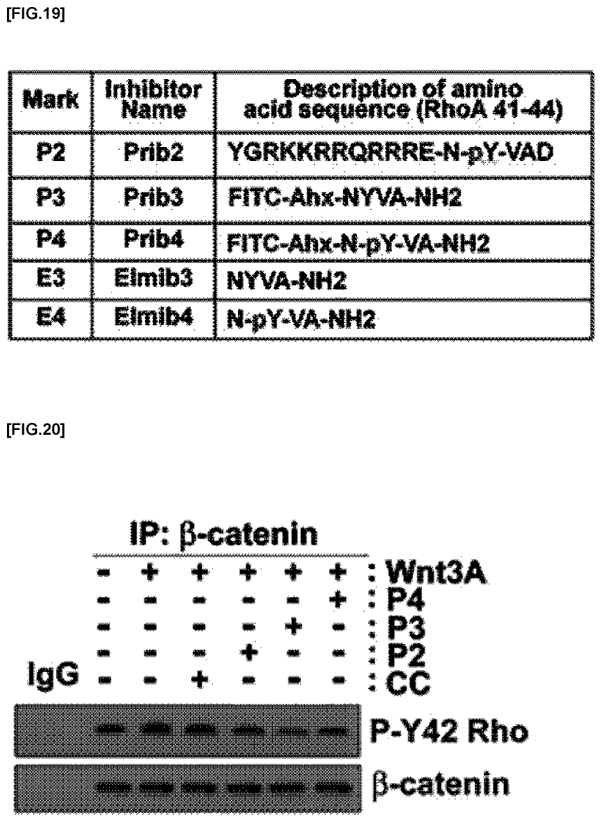

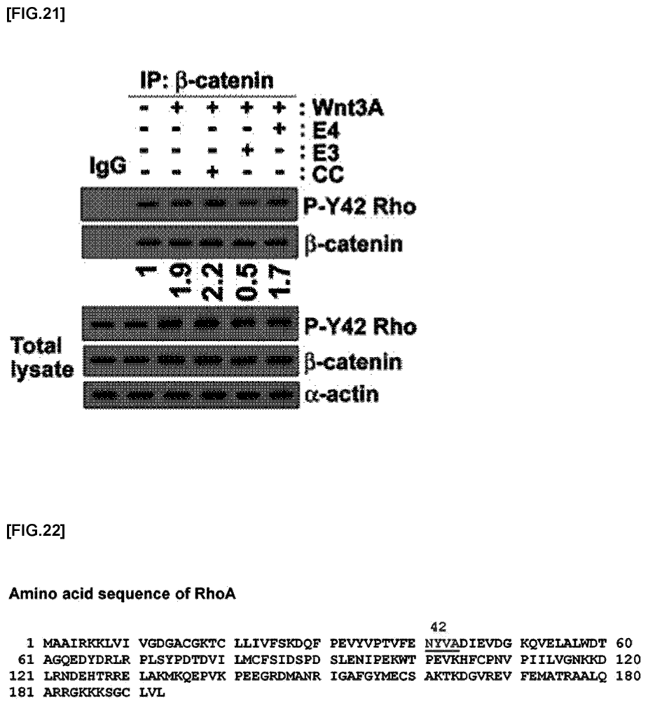

To identify the specific domain of β-catein associated with RhoA, the GST-fusion of the β-catein domains consisting of amino acids (aa) 1-140, 141-390, 391-662, and 663-782 was expressed and purified (FIGS. 15 and 16). While RhoA preloaded with GTPγS can easily bind to aa 1-140 (N-terminal domain, NTD) of the GST-β-catenin domain and conjugated to beads, other domain fusion proteins only slightly bind to RhoA (FIG. 17). In addition, only ectopic expression of NTD (aa 1-140) significantly inhibited cell proliferation due to Wnt3A (FIG. 18). The present inventors have designed a competitive inhibitor that prevents the complex formation between p-Tyr42 RhoA and β-catenin or other effector proteins (FIG. 19). P2, P3 P4, and EM3 inhibited the co-immunoprecipitation of p-Tyr42 RhoA and β-catenin (FIGS. 20 and 21).

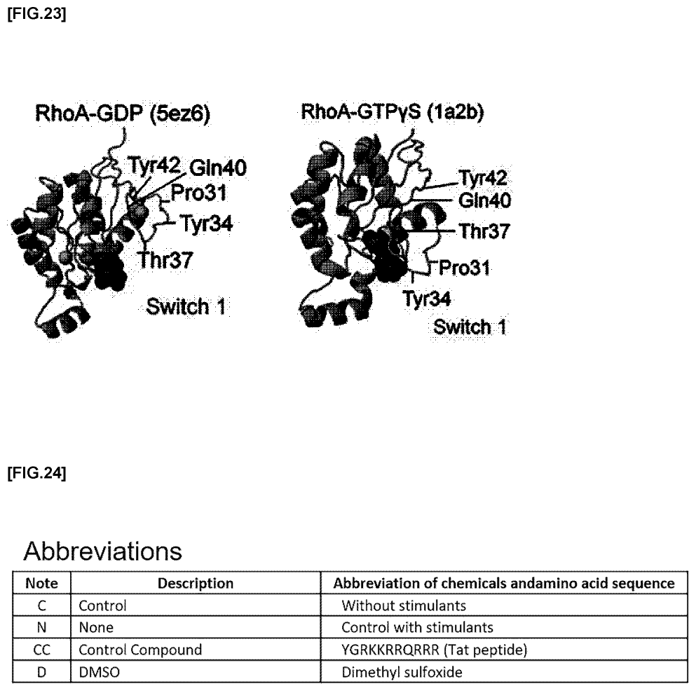

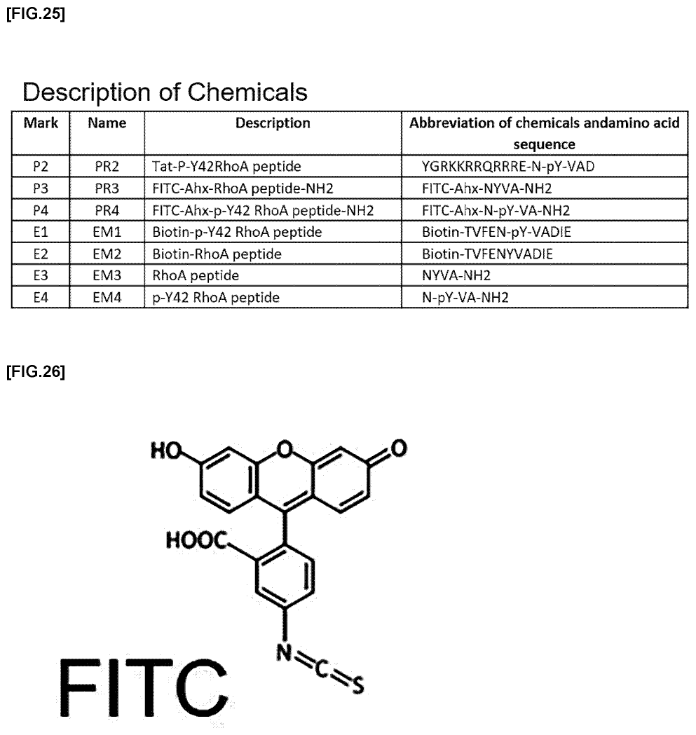

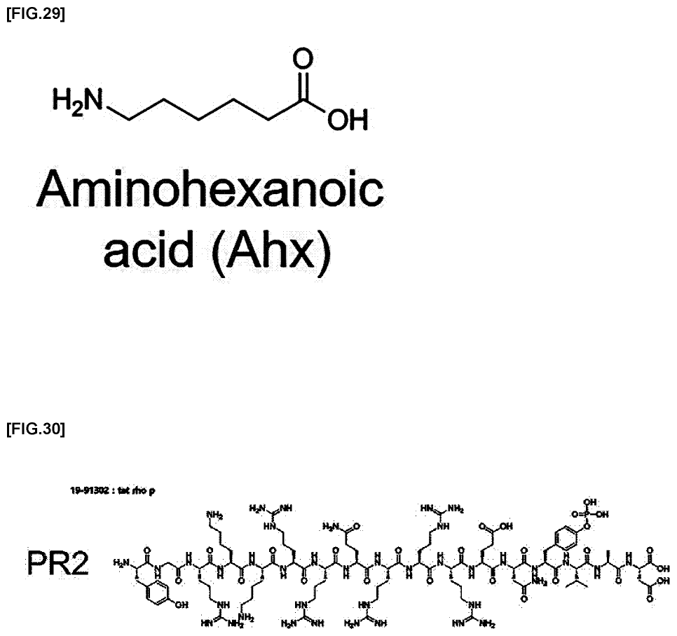

Taken together, it is speculated that inhibition of the p-Tyr42 residue of RhoA could be a good strategy to protect cancer. In particular, the amino acid sequence (FIG. 22) and the 3D structure of RhoA (FIG. 23) are illustrated. The abbreviations of the reagents used in the present disclosure (FIG. 24), RhoA inhibitors (PR2, PR3, PR4, EM1, EM2, EM3, and EM4) (FIG. 25), other chemicals (FIGS. 26 to 29), and chemical structures of PR2, PR3, PR4, EM1, EM2, EM3, and EM4 (FIGS. 30 to 36) are shown, respectively.

Anticancer Drug Containing Peptide of RhoA's p-Tyr42

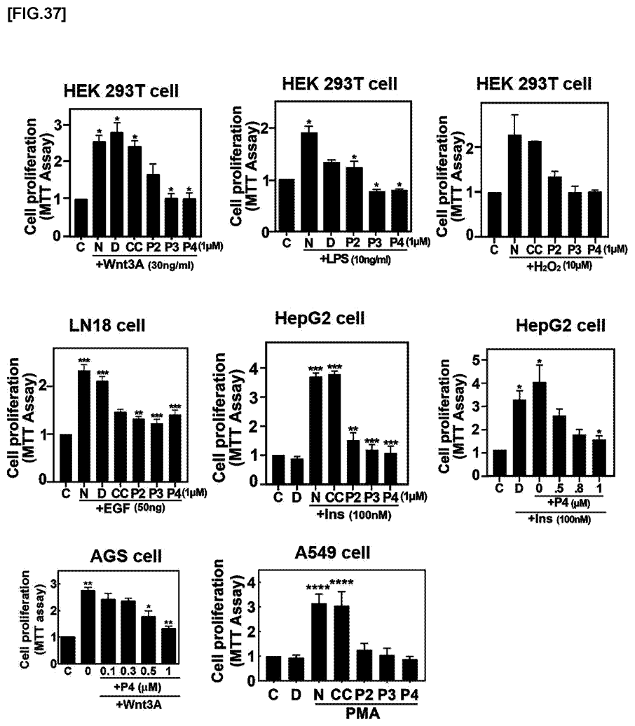

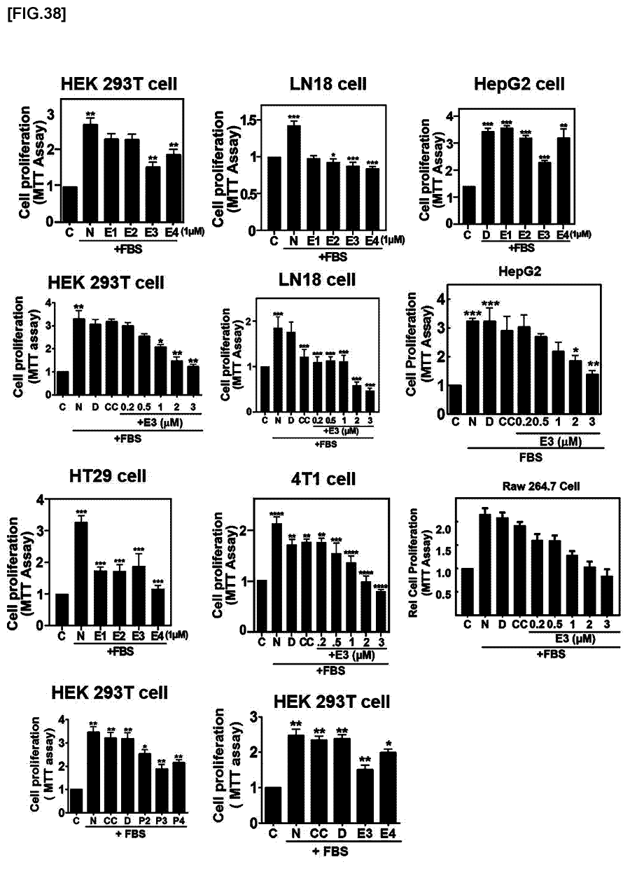

In particular, PR2, PR3, and PR4 significantly inhibited the proliferation of HEK293T cells by Wnt3A, the proliferation of HEK293T cells by LPS, the proliferation of HEk293T cells by H₂O₂, the proliferation of HepG2 cells by insulin, the proliferation of LN18 cells by EGF, and the proliferation of A549 cells by PMA. In addition, 1 μM P4 almost completely inhibited the proliferation of HepG2 cells by insulin, and almost completely inhibited the proliferation of AGS cells by Wnt3A (FIG. 37). Moreover, EM3 (NYVA-NH2) significantly inhibited the proliferation of FBS-stimulated HEK293T, LN18, HepG2, HT29, 4T1, and RAW264.7 cells (FIG. 38). The phosphoric acid group of EM4 has the potential to prevent the movement of the plasma membrane.

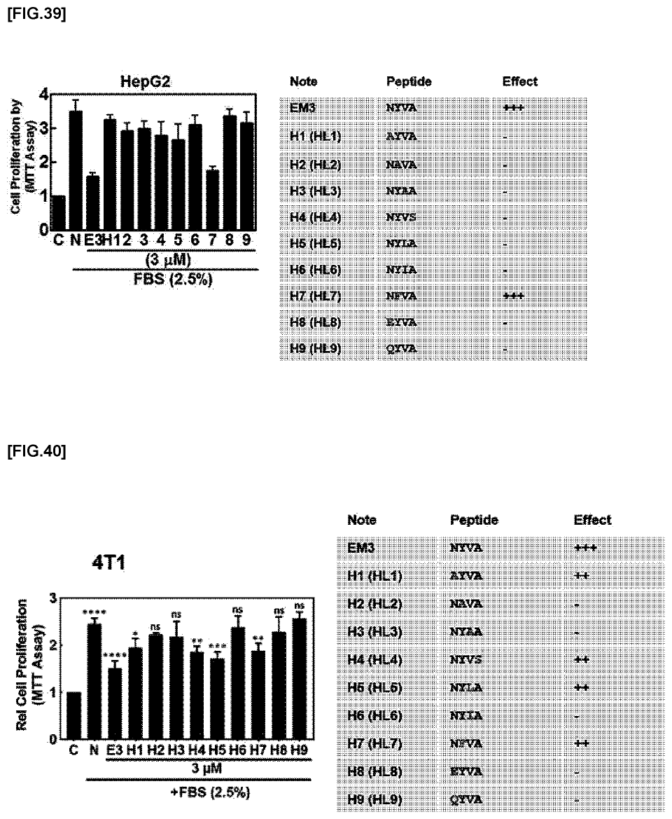

In addition, the present inventors have attempted to examine the amino acid substitution of EM3 for cell proliferation. The NFVA peptide and NYVA showed similar inhibitory effects on proliferation of HepG2 cells (FIG. 39). Likewise, NYVS, LYLA, NFVA, and NYVA showed similar inhibitory effects on proliferation of 4T1 cells (FIG. 40).

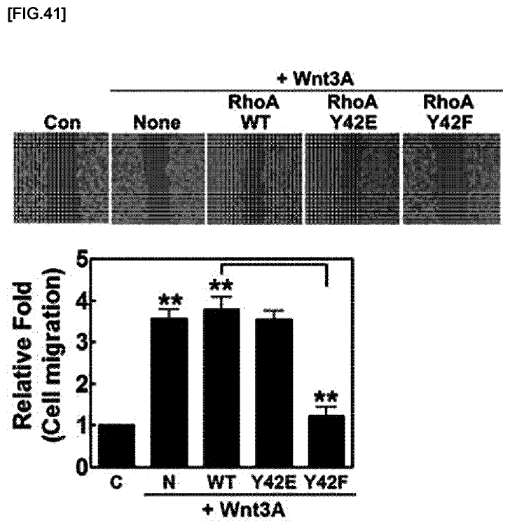

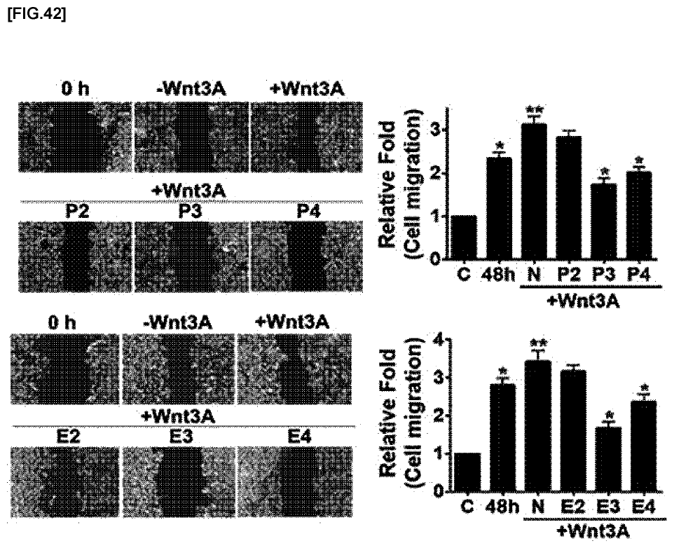

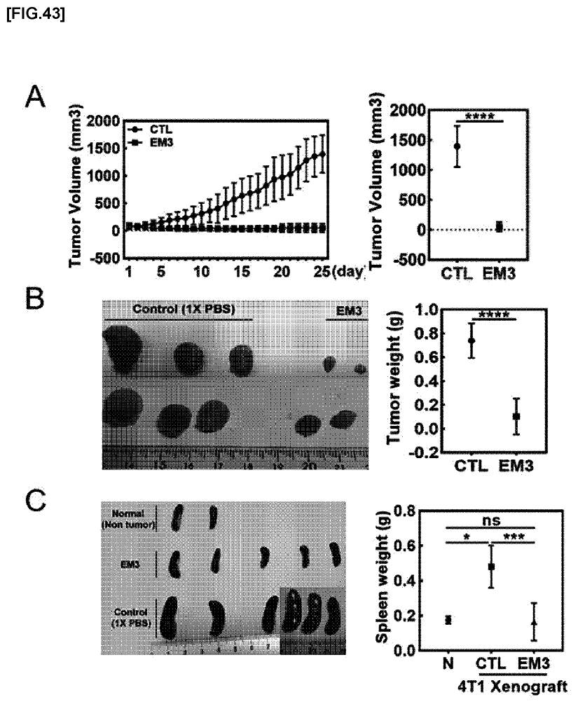

The migration of HEK293T cells, which were transfected with RhoA Y42F (dephospho-mimic) but not with RhoA WT or RhoA Y42E (phospho-mimic), was inhibited in response to Wnt3A, which shows the cell migration of p-Tyr42 RhoA to Wnt3A (FIG. 41). Moreover, PR3, PR4, EM3, and EM4 imply the same by reducing the migration of HEK293 cells in response to Wnt3A (FIG. 42).

Xenotransplantation Experiment

Animal care and experiments were performed by the approval of the Institutional Animal Care and Use Committee (HIRB-2016-52) of Hallym University. Male BALB/c mice (7 weeks old) were injected with 4T1 cells (5×10⁵ cells in 100 pt of 1×PBS), and randomly assigned to an EM3 treatment group (7 mice) or the control group (6 mice). The normal group also included two non-tumor mice. Cells were injected subcutaneously and EM3 (2.5 mM, 10 μL/mouse) and 1× PBS (10 μL/mouse) were applied daily. Tumor volume was measured daily and tumor weight and spleen size were measured after sacrificing the mice.

FIG. 43 shows the results of xenograft experiments measuring (A) the tumor volume in breast cancer cells of a 4T1 mouse during the period of tumor formation, (B) the size and weight of the tumor in breast cancer cells of a 4T1 mouse after sacrifice, and (C) the size and weight of the spleen of the mouse breast cancer cell 4T1 after sacrifice.

It is to be noted that EM3 dramatically decreased tumor volume and tumor weight in xenograft experiments (FIGS. 43A and 43B). While the spleen size was increased in tumor-bearing mice, EM3 treated mice showed the same size compared to control mice (FIG. 43C).

Hereinafter, the drawings attached to the present specification will be described in more detail. First, FIGS. 1 to 4 show clinical results of the expression of p-Tyr42 Rho in cancer patients, and various cancer types of the patient were stained with p-Tyr42 Rho antibody. Various gastric cancer tissues were stained with the p-Tyr42 Rho antibody, and quantitative scoring of gastric cancer tissue stages was performed. The survival probability of gastric cancer patients with low, medium, and high RhoA mRNA levels were obtained from raw data from the Cancer Genome Atlas operated by the National Cancer Institute of the United States. P-Tyr42 RhoA levels in tissue samples of gastric cancer patients were analyzed by Western blotting. Blue star: high level; Red Star: this represents a patient with cancer (death). The band intensity of p-Tyr42 Rho level for gastric cancer patients was plotted against the cancer stage. The patient with red spots died, and the patient with blue spots was alive. Survival probabilities based on the relatively detected p-Tyr42 Rho levels were plotted and displayed. A schematic diagram of Wnt3A signaling through p-Tyr42 RhoA is shown. RhoA can be activated by Wnt3A through Daam, Disheveled, and WGEF. Activated RhoA stimulates superoxide production through p-p47PHOX. Consequently, superoxide inhibits nucleoredoxin which inhibits Dishevelled. Superoxide activates Src kinase, and then induces the phosphorylation of Tyr42 of RhoA and induces the phosphorylation of Tyr216 of GSK-3β. This phosphorylates LRP5/6 to recruite a “destruction box” containing APC, Axin, and GSK-3β, thereby leading to stabilization of β-catenin. P-Tyr42 RhoA binds to the N-terminus of β-catenin and thereby induces nuclear localization of the protein complex. P-Tyr42 RhoA binds to the promoter of the Vim gene along with β-catenin and thereby increases the expression of vimentin, which is a typical marker of EMT.

FIGS. 5 to 10 show that P-Tyr42 RhoA induces cell proliferation in response to Wnt3A, in which (A) HEK293T cells were stimulated with Wnt3A (30 ng/mL) along with DAPI (1 μg/mL) staining for 10 minutes, and DAPI fluorescence was measured with a fluorescence microscope; (B, C) HEK293T (B) cells and RAW264.7 (C) cells were pretreated with Tat-C3 (1 μg/mL) and Y27632 (10 μM) for one hour, followed by treatment with Wnt3A (30 ng/mL) for two days, and cell proliferation was measured under a fluorescence microscope; and (D) HEK293T cells were transfected with RhoA WT, Y42E, and Y42F (4 μg DNA) for 24 hours, then treated with Wnt3A (30 ng/mL) for two days, and MTT assay was used for cell proliferation; (E) HEK293T cells were pretreated with Tat-C3 (1 μg/mL) and Y27632 (10 1.1.1\4) for one hour, and then treated with Wnt3A (30 ng/mL) for two hours, and cyclin D1 and c-Myc were subjected to Western blotting; and (F) HEK293T cells were transfected with RhoA WT, Y42E, and Y42F (4 μg DNA) for 24 hours, and then treated with Wnt3A (30 ng/mL) for two days, and cyclin D1 and c-Myc were subjected to Western blotting.

FIGS. 11 to 21 show the interaction of β-catenin with RhoA in response to Wnt3A, in which (A, B) HEK293T cells were co-immunoprecipitated in cell lysates by treating with Wnt3A (30 ng/mL), β-catenin, or p-Tyr42 Rho, and the immunoprecipitated β-catenin was detected by Western blotting, and the immunoprecipitated β-catenin was subjected to immunoblotting; (C) LPA (10 μM) was treated on HEK293T cells, β-catenin was immunoprecipitated from cell lysates, and the co-immunoprecipitated RhoA and p-Tyr42 RhoA were detected by Western blotting; (D) recombinant purified RhoA was preloaded with GDP or GTPγS at RT for 30 minutes and then incubated with GST-β-catenin (0.1 μg) at RT for two hours, and RhoA relating to GST-β-catenin was detected by Western blotting; (E) a schematic diagram of the β-catenin domain is shown; (F) a recombinant purified domain of β-catenin was subjected to SDS-PAGE and Coomassie blue staining; (G) RhoA relating to the GST-β-catenin domain was detected by Western blotting; (H) RhoA inhibitors were designed; (I) the cells were pretreated with P2, P3, and P4 inhibitors and then treated with Wnt3A, and β-catenin was immunoprecipitated, and the co-immunoprecipitated p-Tyr42 RhoA was detected by immunoblotting; and (J, I) the cells were pretreated with EM3 and EM4 inhibitors and then treated with Wnt3A, and β-catenin was immunoprecipitated and the co-immunoprecipitated p-Tyr42 RhoA was detected by immunoblotting.

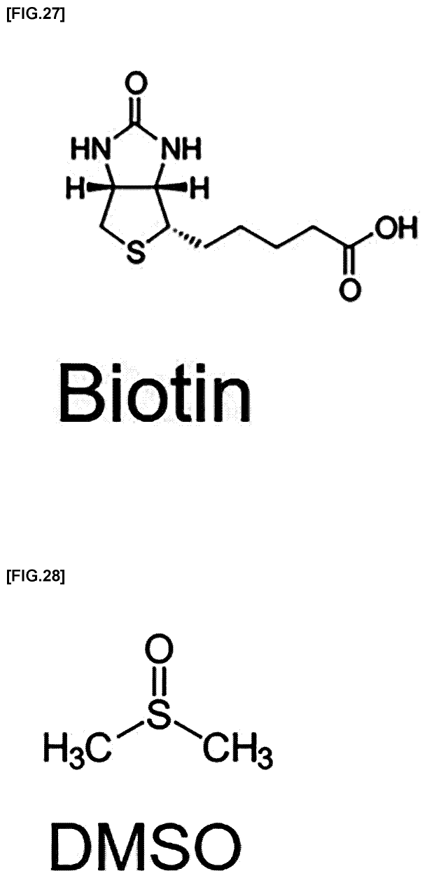

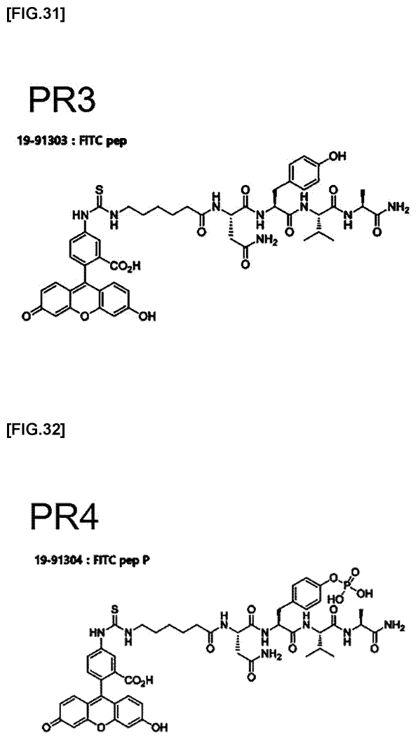

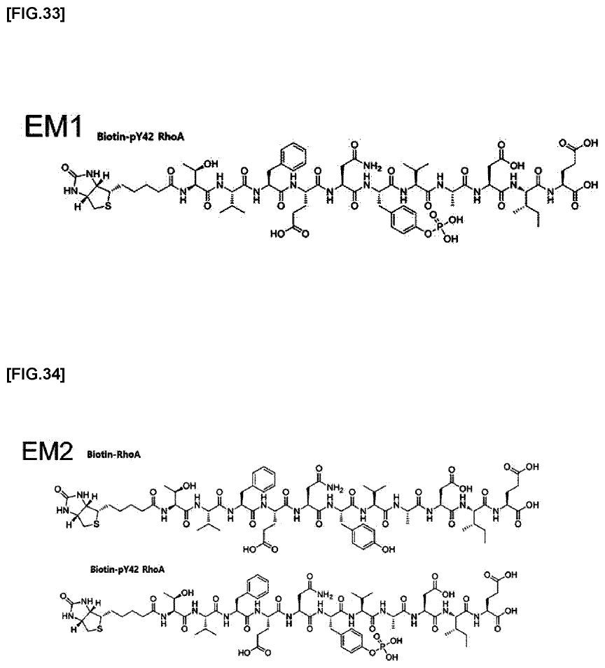

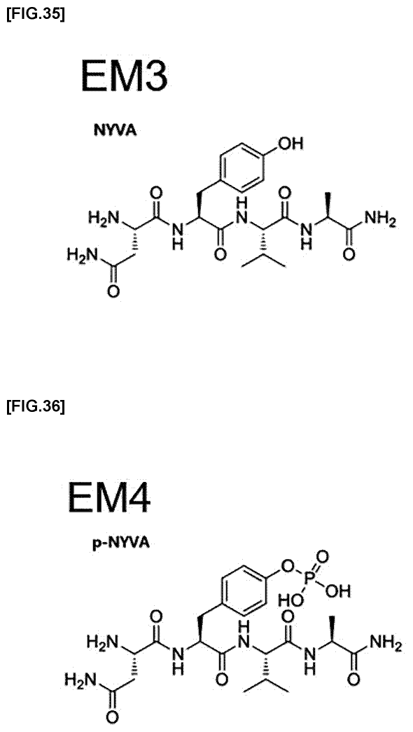

FIGS. 22 to 36 show the designed RhoA inhibitors, in which (FIG. 22) the amino acid sequence of RhoA is presented, and the peptides around Tyr42 of RhoA are shown in blue and red; (FIG. 23) the three-dimensional structure of RhoA bound to GDP or GTPγS is shown; (FIG. 24) in the result, the abbreviation of nots is indicated; (FIG. 25) RhoA peptide inhibitors are described; (FIG. 26) a chemical structure of fluorescein isothiocyanate (FITC); (FIG. 27) a chemical structure of biotin; (FIG. 28) a chemical structure of dimethylsulfoxide (DMSO); (FIG. 29) a chemical structure of aminohexanoic acid; (FIG. 30) a chemical structure of PR2 (P2); (FIG. 31) a chemical structure of PR3 (P3); (FIG. 32) a chemical structure of PR4 (P4); (FIG. 33) a chemical structure of EM1 (E1); (FIG. 34) a chemical structure of EM2 (E2); (FIG. 35) a chemical structure of EM3 (E3); and (FIG. 36) a chemical structure of EM4 (E4).

FIG. 37 shows the inhibition of proliferation of various cancer cells by RhoA peptide inhibitors (PR2, PR3, and PR4), in which RhoA peptide inhibitors were pre-incubated for one hour and then treated with various stimulants, such as Wnt3A, lipopolisaccharide (LPS), hydrogen peroxide, epidermal growth factor (EGF), insulin, and phorbol myristate (PMA), for 48 hours. The MTT assay was performed to measure the proliferation of various cancer cell lines, such as HEK293T, LN18 glioma, HepG2 liver cancer, AGS gastric cancer, and A549 lung cancer cells.

FIG. 38 shows the inhibition of proliferation of various cancer cells by RhoA peptide inhibitors (EM3/E3), in which the cells were pretreated with RhoA peptide inhibitors (EM1, EM2, EM3, and EM4) for one hour and then treated with 2% FBS for 48 hours. The MTT assay was performed to measure the proliferation of various cancer cell lines, such as HEK293T, LN18 glioma, HepG2 liver cancer, HT29 colon cancer, 4T1 mouse breast cancer, and RAW264.7 mouse macrophages.

FIGS. 39 and 40 show the effect of the substituted amino acids of EM3 on cell proliferation, and the present inventors have designed various peptide inhibitors replaced with other amino acids. The cells were cultured in DMEM containing 2.5% FBS for 48 hours and cell proliferation was measured by MTT assay. FIG. 39 shows that HL7 (NFVA) (3 μM) and EM3 showed a similar inhibitory effect on HepG2 cell proliferation. FIG. 40 shows that HL1 (AYVA), HL4 (NYVS), HL5 (NYLA), and HL7 (NFVA) are less effective than EM3 and have an inhibitory effect on HepG2 cell proliferation.

FIGS. 41 and 42 show the prevention of cancer cell migration by the RhoA peptide inhibitor, in which FIG. 41 shows that HEK293T cells were transfected by way of RhoA WT, Y42E (phospho-mimic), and Y42F (dephospho-mimic) mutations and that cell migration was measured by a wound healing method; and FIG. 42 shows that HEK293T cells were pretreated with PR2, PR3, PR4, EM2, EM3, and EM4 (1 μM) and stimulated with Wnt3A (30 ng/ml), in which the cell migration for 48 hours was measured by the wound healing method.

1. A method of treating cancer, the method comprising administering a treatment effective amount of the compound of Formula 1 below as an active ingredient:

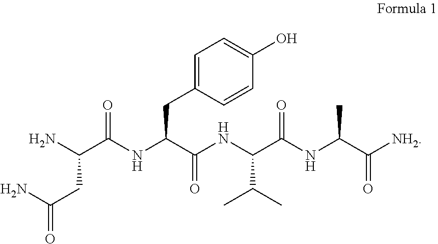 [SMILES: CC(C)[C@H](NC(=O)[C@H](Cc1ccc(O)cc1)NC(=O)[C@@H](N)CC(N)=O)C(=O)N[C@@H](C)C(N)=O]

2. A method of treating cancer, the method comprising administering a treatment effective amount of the compound of Formula 2 below as an active ingredient:

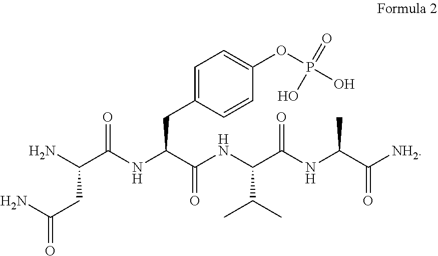 [SMILES: CC(C)[C@H](NC(=O)[C@H](Cc1ccc(OP(=O)(O)O)cc1)NC(=O)[C@@H](N)CC(N)=O)C(=O)N[C@@H](C)C(N)=O]

3. A method of treating cancer, the method comprising administering a treatment effective amount of the compound of Formula 3 below as an active ingredient:

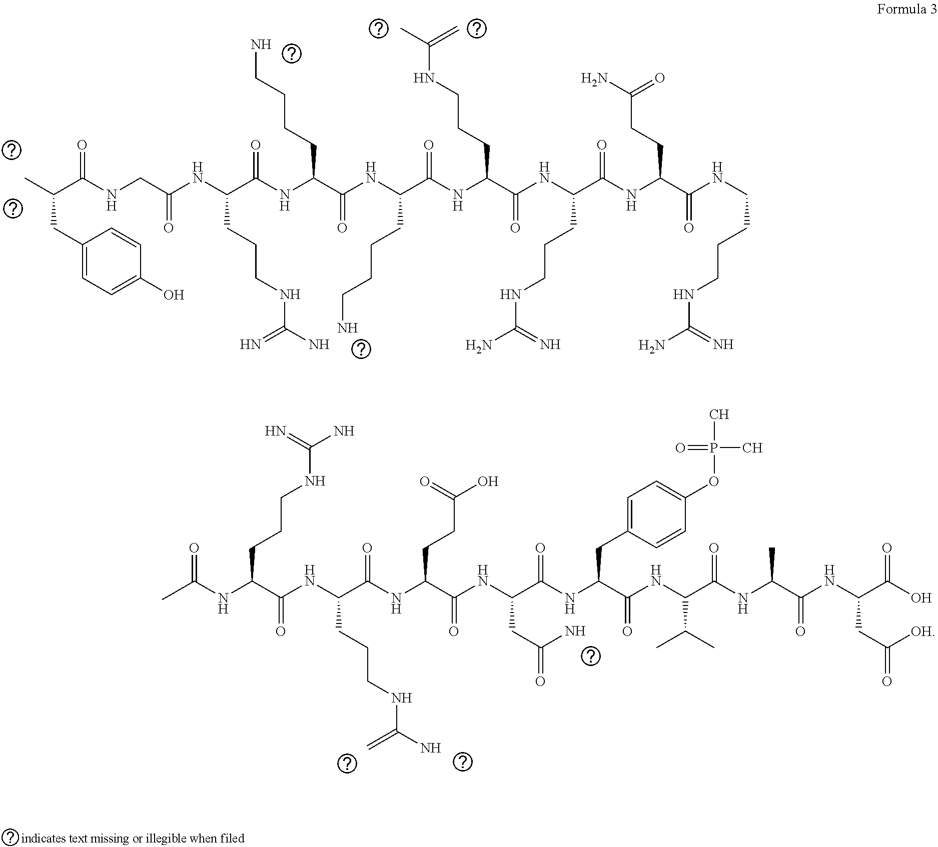

4. A method of treating cancer, the method comprising administering a treatment effective amount of the compound of Formula 4 below as an active ingredient:

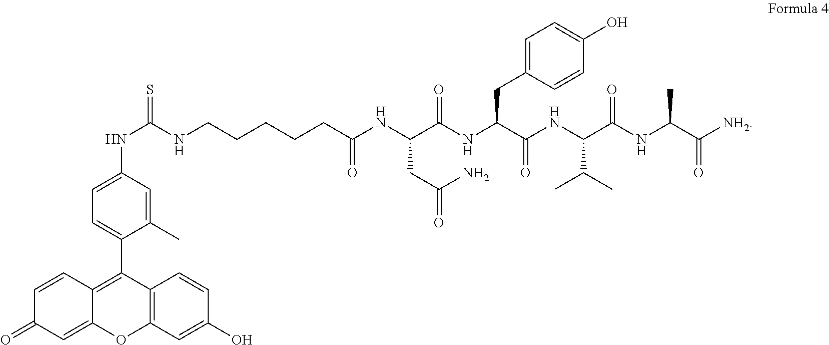 [SMILES: Cc1cc(NC(=S)NCCCCCC(=O)N[C@@H](CC(N)=O)C(=O)N[C@@H](Cc2ccc(O)cc2)C(=O)N[C@H](C(=O)N[C@@H](C)C(N)=O)C(C)C)ccc1-c1c2ccc(=O)cc-2oc2cc(O)ccc12]

5. A method of treating cancer, the method comprising administering a treatment effective amount of the compound of Formula 5 below as an active ingredient:

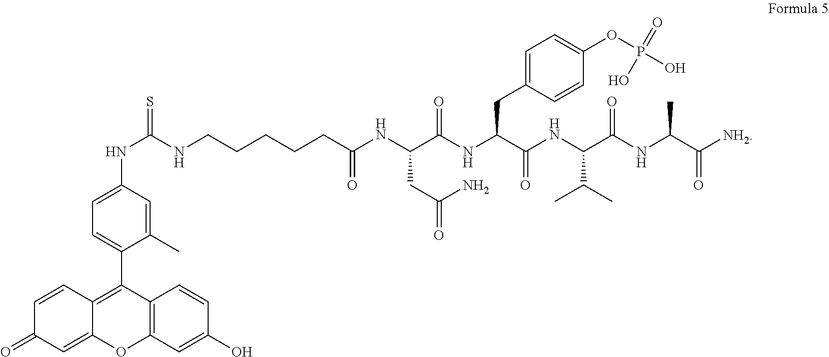 [SMILES: Cc1cc(NC(=S)NCCCCCC(=O)N[C@@H](CC(N)=O)C(=O)N[C@@H](Cc2ccc(OP(=O)(O)O)cc2)C(=O)N[C@H](C(=O)N[C@@H](C)C(N)=O)C(C)C)ccc1-c1c2ccc(=O)cc-2oc2cc(O)ccc12]

6. A method of treating cancer, the method comprising administering a treatment effective amount of the compound of Formula 6 below as an active ingredient:

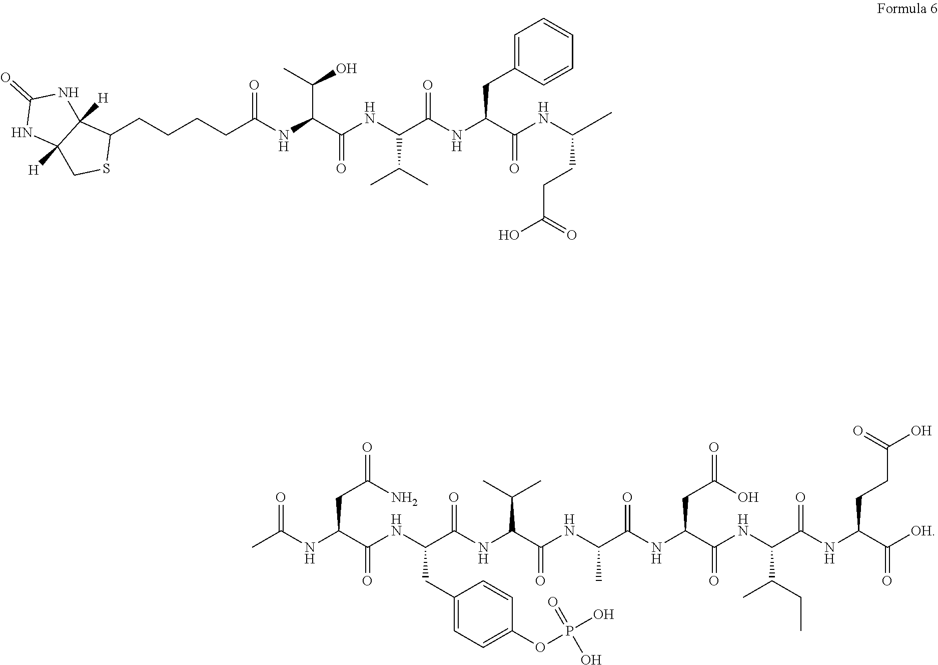

7. A method of treating cancer, the method comprising administering a treatment effective amount of the compound of Formula 6 below as an active ingredient: [chemical structure]


SEQUENCE LISTING (1 sequence)

SEQ ID NO: 1 - protein, 193 aa, Artificial Sequence
  RhoA
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL